(19) United States
(12) Patent Application Publication
(10) Pub. No.: US 2007/0009580 A1
(43) Pub. Date: Jan. 11, 2007

(54) Non-adhesive hydrogels

(76) Inventors: Frank DiCosmo, Ontario (CA); Valerio DiTizio, Ontario (CA)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 11/169,481

(22) Filed: Jun. 29, 2005

Related U.S. Application Data
(60) Provisional application No. 60/583,595, filed on Jun. 30, 2004.

(51) Int. Cl. A61K 9/70; A61K 9/14; A61K 33/38

(52) U.S. Cl. 424/443; 424/484; 424/618

(57) ABSTRACT

Substantially non-adhesive hydrogels are useful as wound dressings, wound barriers, therapeutic drug delivery devices and the like. The substantially non-adhesive hydrogels are synthesized by a method that comprises irradiating a solution comprising a biological polymer that is biodegradable and biocompatible, using ionizing radiation, whereby free radicals of the biological polymer are formed and cross-linking occurs between the biological polymer radicals to provide the hydrogel.

### FIELD OF THE INVENTION

The present invention relates to hydrogels. In particular, the present invention relates to non-adhesive hydrogels and the method of making the same. Such non-adhesive hydrogels are useful as wound dressings, wound barriers, therapeutic drug delivery devices and the like.

### BACKGROUND TO THE INVENTION

Hydrogels are a group of biomaterials that have been used extensively in the medical field as they are gas permeable, biocompatible, biodegradable, cause little inflammation and can be manufactured to be non-toxic to virtually all cells and tissues. Hydrogels are useful as wound dressings, artificial skin, and therapeutic drug delivery devices, whereby the hydrogels can retain therapeutics and deliver such therapeutics to appropriate cells and tissues, as exemplified in Applicant's U.S. Pat. No. 6,475,516.

A hydrogel is any material, which forms, to various degrees, a jelly-like product when suspended in a solvent, typically polar solvents. More specifically, hydrogels are cross-linked hydrophilic polymers, including proteins, such as collagen, gelatin, pectin, cellulose or fractions and derivatives thereof. Constituents such as hemoglobin may also be included in the hydrogel mixture.

Hydrogels may be made using various synthetic routes. In particular, hydrogels may be synthesized from non-biological monomers or macromers using photopolymerization. These hydrogels are good candidates for many medical applications including tissue engineering (Nguyen, K. T., and West, J. L. Photopolymerizable Hydrogels for Tissue Engineering Applications. Biomaterials 23: 4307-4314, 2002), ophthalmic applications and for closing surgical wounds. U.S. Pat. No. 4,871,490 is directed to adhesive hydrogels formed by irradiating synthetic and natural polymers using ionizing gamma irradiation having an energy of 25 to 40 KGy. Yoshi et al. Radiation Physics and Chemistry, 55: 133-138, 1999 utilized electron beam crosslinked polyethylene oxide and polyethylene oxide-polyvinylalcohol blend hydrogels as wound dressings.

Hydrogels for medical applications, including tissue engineering, hemostatic, and wound applications, have generally been formed from macromolecular hydrogel precursors with reactive linking groups. Irradiation of the hydrogel precursors have resulted in the formation of a sticky or adhesive hydrogel, as exemplified for vascular puncture closures, surgical or hemostatic sponges, surgical sealants and flowable hemostatic agents.

Synthesis of antibacterial polyvinylalcohol/carboxymethylated-chitosan blend hydrogels using electron beam irradiation has been described in Zhao, et al. Carbohydrate Polymers, 53: 439-436, 2003. An adhesive wound dressing has also been described in European Patent Application 450671, wherein the wound dressing comprises (1) a lower layer of a hydrogel of a polymer, cross-linked using electron beam radiation, to which one or more medicinal and/or antibacterial agents and/or one or more auxiliary substances may be added, and (2) a polymeric top layer. In practice, the adhesive hydrogel is further bonded to a textile layer, preferably a knitted fabric of a polyester, a polyamide or a polyurethane to provide elasticity and strength. U.S. Pat. No. 5,863,984 describes the use of ionizing radiation for grafting conjugated-collagen biopolymers onto synthetic materials. These materials are intended to be adhesive to mammalian tissue and cells.

Electron beam curing of methacrylated gelatin provides a crosslinked, resilient material with an extremely low oxygen permeability and yields a coating that is an excellent barrier to oxygen transmission. Such materials are excluded from providing wound dressing applications (Scherzer, Nuclear Instruments and Methods in Physics Research B, 131: 382-391, 1997), as they are tough, hard, impervious, and resilient coatings.

In general, hydrogels used as wound dressings cause little inflammation, are biocompatible, oxygen and carbon dioxide transmissible and, notably, are adherent to skin and tissue. There is a need, however, for a less complex, more cost-effective and efficient way of making such hydrogels, in particular, hydrogels made from biological polymers. Presently, in order to obtain hydrogels from biological polymers, such as gelatin (denatured collagen), the biological polymers are modified prior to polymerization in order to provide a hydrogel that is stable at temperatures of at least body temperature (37° C.) such that it does not melt during use or during shipping and storage at elevated temperatures.

There is a need, therefore, for improved hydrogels that can be used as or in wound dressings, therapeutic drug delivery devices, wound barriers and the like to reduce chronic inflammation and hydrate and promote a moist wound environment. There is also a need for an improved hydrogel that is stable and substantially non-adhesive. Such non-adhesive hydrogels may be especially useful as wound dressings for damaged tissue, such as burn wounds and also sensitive regenerating tissues that should not be exposed to an adhesive or sticky material.

### SUMMARY OF THE INVENTION

The invention is directed to novel substantially non-adhesive hydrogels and methods for making such hydrogels. The substantially non-adhesive hydrogels may be used as, but not limited to, wound barriers, wound dressings, and in therapeutic drug, medicament and/or chemical agent delivery.

In accordance with one aspect of the present invention, there is provided a method for synthesizing a substantially non-adhesive hydrogel, the method comprising: irradiating a solution comprising a biological polymer that is biodegradable and biocompatible, using ionizing radiation, whereby free radicals of the biological polymer are formed and cross-linking occurs between the biological polymer radicals to provide the hydrogel.

In accordance with another aspect of the present invention, there is provided a method for synthesizing a substantially non-adhesive hydrogel, the method comprising: irradiating a solution comprising a polar solvent and a biological polymer that is biodegradable and biocompatible, using ionizing radiation, whereby free radicals of the biological polymer are formed and cross-linking occurs between the biological polymer radicals to provide the hydrogel.

In accordance with yet another aspect of the present invention, there is provided a substantially non-adhesive hydrogel, the hydrogel being made by a method comprising: irradiating a solution comprising a biological polymer that is biodegradable and biocompatible, using ionizing radiation, whereby free radicals of the biological polymer are formed and cross-linking occurs between the biological polymer radicals to provide the hydrogel; and isolating the hydrogel.

In accordance with certain aspects of the present invention, the ionizing radiation is electron beam radiation.

In accordance with another aspect of the present invention, there is provided a method for synthesizing a substantially non-adhesive hydrogel, the method comprising: irradiating a solution comprising a biological polymer that is biodegradable and biocompatible, using from about 5 KGy to about 50 KGy electron beam radiation, whereby free radicals of the biological polymer are formed and cross-linking occurs between the biological polymer radicals to provide the hydrogel.

In accordance with another aspect of the present invention, there is provided a method for synthesizing a substantially non-adhesive hydrogel, the method comprising: irradiating a solution comprising a polar solvent and a biological polymer that is biodegradable and biocompatible, using from about 5 KGy to about 50 KGy using electron beam radiation, whereby free radicals of the biological polymer are formed and cross-linking occurs between the biological polymer radicals to provide the hydrogel.

In accordance with yet another aspect of the present invention, there is provided a substantially non-adhesive hydrogel, the hydrogel being made by a method comprising: irradiating a solution comprising a biological polymer that is biodegradable and biocompatible, using from about 5 KGy to about 50 KGy using electron beam radiation, whereby free radicals of the biological polymer are formed and cross-linking occurs between the biological polymer radicals to provide the hydrogel; and isolating the hydrogel.

### DETAILED DESCRIPTION OF THE EMBODIMENTS

The invention is directed to novel substantially non-adhesive hydrogels and methods for making such hydrogels.

With respect to the substantially non-adhesive hydrogels of the present invention, the term “substantially non-adhesive” may be understood, in relative terms, to mean a hydrogel that can be applied to damaged tissue, such as burn wounds, and sensitive regenerating tissues such that it is readily removable from the skin without causing further damage to the tissue. In spite of this definition, however, the applicability of the substantially non-adhesive hydrogels of the present invention are not to be limited in any way to damaged tissue and sensitive regenerating tissues.

The substantially non-adhesive hydrogels can be synthesized using the method of the present invention without having to incorporate any cross-linking agent(s). In one embodiment of the invention, the substantially non-adhesive hydrogel is made by irradiating a solution using ionizing radiation. The solution includes a biological polymer that is biodegradable and biocompatible. The solution may also include a polar solvent.

In further embodiments, when making the solutions of the biological polymer, the biological polymer is mixed with a particular solvent and heated to dissolve the polymer. The solution is poured into a mold, such as a polystyrene dish, or simply poured onto a surface, and is subsequently, allowed to solidify, for example, at room temperature. The mold or surface containing the solution is then irradiated.

The substantially non-adhesive hydrogels of the present invention can absorb significant amounts of fluid or exudate emanating from a wound or other skin surface abrasion. It is known that the accumulation of excess wound exudates is detrimental to healing and provides a fertile site for the growth of bacteria which further inhibits the healing process. Due to the absorbency of the hydrogels, the change of wound dressings can occur less frequently and still retain a sterile environment. Of course, the wound dressing can be changed as needed if exudate production is high.

The substantially non-adhesive hydrogels can maintain the wound in a moist condition, which not only facilitates healing but also enhances the cosmetic appearance of the wound as it heals. Furthermore, these specific hydrogels can be used as, but not limited to, wound barriers, wound dressings, and in therapeutic drug, medicament and/or chemical agent delivery devices to deliver medicaments to, for example, the surface of skin, damaged tissue, sensitive regenerating tissues, exit sites of medical devices, the internal mucosa, tissues and organs of mammals, such as humans.

The polar solvent for use in the present invention may include any suitable polar solvent, as is understood by one skilled in the art. In embodiments, the polar solvent may be selected from, but not limited to, water and/or lower alcohols, such as C₁ to C₄ alcohols (e.g. methanol and ethanol).

Irradiation of the solution of the present invention may be achieved using ionizing radiation. Typically, irradiation of the solution is achieved using electron beam radiation. Any electron beam source known to those skilled in the art may be used. Without being limited thereto, an example of a convenient electron beam source is from Dynamitron™ instrument Model 1500-40 manufactured by Radiation Dynamics, Inc.

In some embodiments, the electron beam radiation dose is from about 5 KGy to about 50 KGy, specifically from about 5 KGy to about 40 KGy, from about 5 KGy to less than about 40 KGy, from about 15 KGy to about 25 KGy, and more specifically from about 10 KGy to about 20 KGy. Irradiation occurs for a time sufficient such that cross-linking of the biological polymer is substantially complete. In certain embodiments, the amount of residual initial polymer (after irradiation) is less than about 3% for good biocompatibility. Typical times for irradiation include, but are not limited to, from about 1 to about 10 seconds, specifically, from about 2 to about 3 seconds. For example, irradiation of about 20% by weight gelatin solutions can be irradiated for such time periods.

Without being bound by theory, it is believed that the biological polymer absorbs the ionizing radiation and cleaves a carbon-carbon bond, such as adjacent CH₂ groups on neighboring polyamino molecules, or one of the CH₂ groups may lose a proton to yield CH radicals that cross-link to form new carbon-carbon bonds to ultimately provide the hydrogel of the present invention.

The biological polymer may be any biodegradable and biocompatible polymer. In embodiments, the polymers are chosen from proteins and carbohydrates. In particular, the polymers may be selected from, but not limited to, collagen, hemoglobin, gelatin, pectin, cellulose, derivatives thereof and mixtures thereof. The proteins, such as gelatin, may be modified or unmodified.

In embodiments, the amount of biological polymer(s) used can be from about 10 to about 50% by weight based on the total weight of solution, about 10 to about 45% by weight, or about 15 to about 30% by weight.

In embodiments, the resultant substantially non-adhesive hydrogel comprises from about 1% to about 50% by weight of the cross-linked biological polymer based on the total hydrogel weight, typically, about 20% by weight of the cross-linked biological polymer.

When using the substantially non-adhesive hydrogels as wound dressings, the gel may also contain a buffer system to help inhibit discoloration and/or help inhibit breakdown due to the extended presence of water (i.e. help inhibit hydrolysis). Buffers, if used, may be added to the mixture prior to or after curing. Typically, buffers are added to the mixture prior to irradiation. Suitable buffers include, but are not limited to, sodium potassium tartarate, and/or sodium phosphate monobasic (both of which are commercially available from Aldrich Chemical Co., IN.). The use of a buffer system with the present non-adhesive hydrogel can further extend the shelf-life of the hydrogel without discoloration.

The method for synthesizing the substantially non-adhesive hydrogel may further include washing the resultant substantially non-adhesive hydrogel with water and/or a salt solution. The salt solution may be made from any biologically compatible salt, such as ammonium bicarbonate or sodium chloride. In a specific embodiment, the concentrations of these solutions are iso-osmotic relative to physiological saline solutions (0.85%).

The substantially non-adhesive hydrogel of the present invention may be used for at least one of reducing chronic inflammation, absorbing exudates and promoting a moist wound environment. Covering(s), such as wound barrier(s), wound dressing(s), and combinations thereof, may comprise these substantially non-adhesive hydrogel(s). In order to treat a wound, the covering is simply applied to the wound.

To maintain or promote sterility and enhance healing, other additives, such as a therapeutic drug, a medicament and/or a chemical agent, may also be included in the substantially non-adhesive hydrogels before and/or after irradiation (i.e. pharmaceuticals, disinfectants, humectants, plasticizers, etc.). The appropriateness of such additives is generally dependent upon which dressings are to be formulated and applied to a wound. These substantially non-adhesive hydrogels may deliver the therapeutic drug, the medicament and/or the chemical agent to the surface of tissue. Such hydrogels may also be used to deliver the therapeutic drug, the medicament and/or the chemical agent to the surface of intact skin for at least one of exfoliation and treatment of age related conditions in mammals. Covering(s), such as wound barrier(s), wound dressing(s), and combinations thereof, may comprise these substantially non-adhesive hydrogel(s).

In other embodiments, devices incorporating the substantially non-adhesive hydrogel of the present invention, such as a therapeutic drug delivery device, a medicament delivery device and a chemical agent delivery device, may also be used to deliver a therapeutic drug, a medicament and/or a chemical agent. One such device is an occlusive device, which comprises an occlusive structure and the substantially non-adhesive hydrogel. The hydrogel has opposing surfaces such that one surface of the hydrogel is affixed to one surface of the occlusive structure with the other surface of the hydrogel adapted to cover and be in contact with the tissue. The substantially non-adhesive hydrogel of the occlusive device may optionally comprise the therapeutic drug, the medicament and/or the chemical agent.

Silver salts and other medicaments may also be added to the solution during synthesis of the non-adhesive hydrogels. Silver salts, such as silver lactate, may be added such that the non-adhesive hydrogels comprise photoreduced silver and the hydrogel acts as a substantially non-adhesive antimicrobial carrier that can be applied to the surface of tissues and wounds, such as burns, damaged skin and tissues. In other words, the hydrogel acts as a barrier to microbes and contaminants and/or for delivering photo-reduced silver to the surface of a wound to inhibit microbial contamination and infection. When medicaments are not affected by the irradiation process, the medicaments may be incorporated into the mixture prior to irradiation. For instance, the non-adhesive hydrogel incorporating a medicament may be synthesized by irradiating a solution comprising a polar solvent, a biological polymer, and a silver salt.

Alternatively, the medicaments, including silver salts, therapeutics, hormones, vitamins, mixtures thereof and a plurality of other compounds used in medicine and the cosmetic industry may be incorporated into the hydrogel after irradiation. The medicaments may be in solution and/or encapsulated within liposomes.

In embodiments, an effective amount of at least one of a therapeutic drug, a medicament and a chemical agent can be added before and/or after irradiation. The “effective amount” is any amount that provides the therapeutic, medicated, and/or chemical effect. The effective amount may be, for example, 0.1 to 10% by weight based on the total weight of the solution or 0.1 to 1% by weight based on the total weight of the solution.

The substantially non-adhesive hydrogels may also be prepared with a physical support structure to better retain the hydrogel over a wound. This physical support structure may be in the form of an occlusive device having an impermeable backing, i.e. a patch. The non-adhesive hydrogels can also be formed around a web or fibril support and fashioned by cutting into suitable sizes in both surface area and depth, i.e. sheets, strips, squares, circles, ovals, etc.

The above disclosure generally describes particular embodiments of the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

TABLE 1

| | Weight % | Weight/Unit Dressing (g) |
|---|---|---|
| Porcine Gelatin (300 Bloom) | 20 | 2.06 |
| Water | 80 | 8.26 |

The components and amounts used to make a substantially non-adhesive hydrogel are provided in Table 1. A sufficient amount of gelatin was added to water at room temperature (about 22° C.) or at a lower temperature to provide a 20% by weight suspension of gelatin. The gelatin suspension was stirred and heated to about 40° C. until the solids were dissolved. The mixture was then poured into a mold (e.g. polystyrene dish) and allowed to solidify at room temperature for approximately 30 minutes. The mold containing the mixture was placed into the electron beam apparatus (e.g. a Dynamitron™ instrument Model 1500-40 manufactured by Radiation Dynamics, Inc.) and irradiated for about 2 to about 3 seconds at about 15 KGy.

### Example 2

TABLE 2

| | Weight % | Weight/Unit Dressing (g) |
|---|---|---|
| Porcine Gelatin (300 Bloom) | 19.9 | 2.06 |
| Sodium Chloride | 0.06 | 0.006 |
| Silver Lactate | 0.19 | 0.02 |
| Water | 79.8 | 8.26 |

The components and amounts used to make a substantially non-adhesive hydrogel are provided in Table 2. A 10 mM aqueous solution of silver lactate was prepared. A sufficient amount of gelatin was added to the silver lactate solution at room temperature (about 22° C.) or at a lower temperature to provide a 20% by weight suspension of silver/gelatin. The suspension was stirred and heated to about 40° C. until the solids were dissolved. Sodium chloride crystals were then added to the silver/gelatin mixture in order to obtain a solution that was 10 mM in sodium chloride. The mixture was then poured into a mold (e.g. polystyrene dish) and allowed to solidify at room temperature for approximately 30 minutes. The mold containing the mixture was placed into the electron beam apparatus (e.g. a Dynamitron™ instrument Model 1500-40 manufactured by Radiation Dynamics, Inc.) and irradiated for about 2 to about 3 seconds at about 15 KGy.

### Example 3

### Heat Stability of Electron Beam Cross-Linked Hydrogels

The effectiveness of electron beam cross-linking was evaluated by determining the stability of samples incubated at about 37° C. for 24 hours. It is noted that non-cross-linked gelatin hydrogels were unstable at 37° C. and would completely dissolve within seconds. The procedure for determining the heat stability was as follows:
1. Accurately weighed a portion of the hydrogel in a pre-weighed glass vial.
2. Added 15 ml of water to each vial.
3. Incubated at about 40° C. for about 24 hours.
4. Emptied water from the vial and oven-dried the vial containing the hydrogel at about 100° C. overnight.
5. Weighed vials containing hydrogel again.
6. Calculated heat stability expressed as a percentage of weight remaining after hot water treatment.

All samples, regardless of radiation dose or the presence of silver, remained essentially intact throughout the assay. The data in Table 3 demonstrates that all samples retained greater than 50% of their original weight, which indicates that substantial cross-linking of gelatin chains had occurred during the electron beam exposure. Despite the nearly identical stability values, the 15 KGy (1.5 Mrad) exposed samples did swell to a greater extent than did the 20 KGy (2.0 Mrad) exposed samples suggesting that fewer cross-links may be present in the latter material.

TABLE 3

| Sample | Wₛ (g) | W_{s′} (g) | Stability (%) |
|---|---|---|---|
| 20 KGy without Ag | 0.199 | 0.106 | 53.3 |
| 20 KGy with Ag | 0.248 | 0.134 | 56.2 |
| 15 KGy without Ag | 0.238 | 0.130 | 54.8 |
| 15 KGy with Ag | 0.278 | 0.153 | 54.8 |

Note:
1. Wₛ = initial sample dry weight
2. W_{s′} = dry weight after 24 hour incubation at about 40° C.
3. Stability = (Wₛ/W_{s′}) × 100

We claim:

1. A method for synthesizing a substantially non-adhesive hydrogel, the method comprising:
irradiating a solution comprising a biological polymer that is biodegradable and biocompatible, using ionizing radiation, whereby free radicals of the biological polymer are formed and cross-linking occurs between the biological polymer radicals to provide the hydrogel.

2. The method of claim 1, wherein the ionizing radiation is electron beam radiation.

3. The method of claim 2, wherein from about 5 KGy to about 50 Kgy of the electron beam radiation is used.

4. The method of claim 3, wherein from about 15 KGy to about 25 Kgy of the electron beam radiation is used.

5. The method of claim 4, wherein from about 10 KGy to about 20 Kgy of the electron beam radiation is used.

6. The method of claim 1, wherein the biological polymer is selected from the group consisting of proteins, carbohydrates and mixtures thereof.

7. The method of claim 6, wherein the biological polymer is selected from the group consisting of collagen, hemoglobin, gelatin, pectin, cellulose, derivatives thereof and mixtures thereof.

8. The method of claim 7, wherein the biological polymer is gelatin, the gelatin being unmodified.

9. The method of claim 1, wherein the biological polymer is from about 10% to about 50% by weight based on the total weight of solution.

10. The method of claim 9, wherein the biological polymer is from about 10% to about 45% by weight based on the total weight of solution.

11. The method of claim 10, wherein the biological polymer is from about 15% to about 30% by weight based on the total weight of solution.

12. The method of claim 1, wherein the solution further comprises a polar solvent.

13. The method of claim 12, wherein the polar solvent is at least one of water and a C₁ to C₄ alcohol.

14. The method of claim 1, further comprising adding at least one of a therapeutic drug, a medicament and a chemical agent before and/or after irradiation.

15. The method of claim 14, wherein said at least one of the therapeutic drug, the medicament and the chemical agent are selected from the group consisting of silver salts, hormones, vitamins, pharmaceuticals, disinfectants, humectants, and mixtures thereof.

16. The method of claim 14, wherein said at least one of the therapeutic drug, the medicament and the chemical agent is encapsulated within a liposome.

17. The method of claim 1, further comprising adding a silver salt to the solution before irradiation.

18. The method of claim 17, wherein the silver salt is silver lactate.

19. The method of claim 1, wherein the solution further comprises at least one of a buffer and a base.

20. The method of claim 1, further comprising washing the substantially non-adhesive hydrogel with at least one of water and a salt solution.

21. The method of claim 1, further comprising adding the solution to a mold or a surface prior to irradiation.

22. The method of claim 21, further comprising adding the solution to the mold or the surface and allowing the solution to solidify prior to irradiation.

23. A substantially non-adhesive hydrogel made by the method of claim 1.

24. The hydrogel of claim 23, wherein the hydrogel comprises from about 1% to about 50% by weight of a cross-linked biological polymer based on the total weight of the hydrogel.

25. The hydrogel of claim 23, wherein the hydrogel is formed around a web or fibril support.

26. An occlusive device comprising an occlusive structure and the hydrogel of claim 23, wherein the hydrogel has opposing surfaces such that one surface of the hydrogel is affixed to one surface of the occlusive structure with the other surface of the hydrogel being adapted to cover and be in contact with tissue.

27. A covering for at least one of reducing chronic inflammation, absorbing exudates and promoting a moist wound environment, the covering comprising the hydrogel of claim 23.

28. The covering of claim 27, wherein the covering is selected from the group consisting of a wound barrier, a wound dressing, and combinations thereof.

29. A substantially non-adhesive hydrogel made by the method of claim 14.

30. A covering for delivering said at least one of the therapeutic drug, the medicament and the chemical agent to the surface of tissue, the covering comprising the hydrogel of claim 29.

31. The covering of claim 30, wherein the covering delivers said at least one of the therapeutic drug, the medicament and the chemical agent to the surface of intact skin for at least one of exfoliation and treatment of age related conditions in mammals.

32. The covering of claim 30, wherein the covering is selected from the group consisting of a wound barrier, a wound dressing, and combinations thereof.

33. At least one of a therapeutic drug delivery device, a medicament delivery device and a chemical agent delivery device, each comprising the hydrogel of claim 29.

34. A method for treating a wound with a covering comprising the hydrogel of claim 23, the method comprising applying the covering to the wound.

35. The method of claim 34, wherein the covering acts as a barrier to microbes and contaminants.

36. The method of claim 34, wherein the covering is selected from the group consisting of at least one wound barrier, at least one wound dressing, and combinations thereof.

37. A method for treating a wound with a covering comprising the hydrogel of claim 29, the method comprising applying the covering to the wound, wherein said at least one of the therapeutic drug, the medicament and the chemical agent is delivered to the wound.

38. The method of claim 37, wherein the hydrogel comprises photo-reduced silver, the photo-reduced silver being delivered to the surface of the wound to inhibit microbial contamination and infection.

39. The method of claim 37, wherein the covering is selected from the group consisting of a wound barrier, a wound dressing, and combinations thereof.

40. A method for treating tissue with a covering comprising the hydrogel of claim 29, the method comprising applying the covering to the tissue, wherein said at least one of the therapeutic drug, the medicament and the chemical agent is delivered to the tissue for at least one of exfoliation and treatment of age related conditions in mammals.

41. A substantially non-adhesive hydrogel, the hydrogel being made by a method comprising:
irradiating a solution comprising a biological polymer that is biodegradable and biocompatible, using ionizing radiation, whereby free radicals of the biological polymer are formed and cross-linking occurs between the biological polymer radicals to provide the hydrogel; and
isolating the hydrogel.

42. The method of claim 41, wherein the ionizing radiation is electron beam radiation.
